(19) United States
(12) Patent Application Publication
(10) Pub. No.: US 2009/0275966 A1
(43) Pub. Date: Nov. 5, 2009

(54) Flexible inner members having flexible regions comprising a plurality of intertwined helical cuts

(76) Inventor: Miroslav Mitusina, Ruskin, FL (US)

Correspondence Address: EPSTEIN & GERKEN, 1901 RESEARCH BOULEVARD, SUITE 340, ROCKVILLE, MD, 20850, US

(21) Appl. No.: 12/151,277

(22) Filed: May 5, 2008

(51) Int. Cl. A61B 17/32

(52) U.S. Cl. 606/171

(57) ABSTRACT

A flexible inner member for rotation within an angled outer tubular member of a rotary tissue cutting instrument to cut anatomical tissue includes a flexible region for conforming to the configuration of an angled region of the outer tubular member and formed of a plurality of intertwined helical cuts in a cylindrical wall of a tubular body of the inner member. The helical cuts are congruent but differ by a translation or offset along a central longitudinal or helix axis of the tubular body. The helical cuts result in formation of a plurality of intertwined helical strip members in the cylindrical wall, each having opposed ends unified integrally and unitarily or monolithically with the cylindrical wall.

### BACKGROUND OF THE INVENTION

1. Field of the Invention

The present invention pertains generally to flexible inner members rotatably disposed within angled outer tubular members of rotary surgical tissue cutting instruments in which the inner member has a flexible region to transmit torque while conforming to the angled configuration of the outer tubular member. More particularly, the present invention pertains to flexible inner members of angled rotary tissue cutting instruments wherein the flexible region comprises helical cuts formed in a tubular body of the inner member.

2. Brief Discussion of the Related Art

Angled rotary tissue cutting instruments have become widely accepted for use in various surgical procedures to cut anatomical tissue at a surgical site within a patient's body. Angled rotary tissue cutting instruments typically comprise an elongate angled outer tubular member and an elongate flexible inner tubular member rotatably disposed within the angled outer tubular member. A cutting element at a distal end of the inner member is exposed from an opening at a distal end of the outer member to cut anatomical tissue at the surgical site when the inner member is rotated within the outer member. The inner member is ordinarily rotated within the outer member via a powered surgical handpiece coupled to proximal ends of the outer and inner members, with the handpiece being maintained externally of the patient's body. The outer tubular member is formed with one or more angled, curved or bent regions along its length to provide an angled configuration that facilitates positioning of the cutting element at the surgical site when the instrument is introduced in the patient's body, and particularly when the instrument is introduced through a narrow or small size, natural or artificially created entry opening in the patient's body. The inner tubular member is provided with one or more flexible regions to reside within the one or more angled, curved or bent regions of the outer member for transmitting torque to rotate the cutting element while conforming to the angled configuration of the outer member. The angled configuration of the outer member is particularly beneficial in facilitating positioning of the cutting element at the surgical site where there is a non-straight path in the body from the entry opening to the surgical site. In such cases, angled rotary tissue cutting instruments are usually better suited to access the surgical site more easily and quickly, and with less trauma to the patient, than are rotary tissue cutting instruments in which the outer tubular member is longitudinally straight. In many surgical procedures performed using rotary tissue cutting instruments, the internal lumen of the inner tubular member is used to transmit suction to the surgical site to aspirate anatomical tissue and/or fluid through the inner member. In addition, an annular gap or clearance between the internal diameter of the outer member and the external diameter of the inner member is commonly used as an irrigation passage to supply irrigation fluid to the surgical site.

One design advantage in rotary tissue cutting instruments is to minimize the external diametric size of the outer member to allow introduction of the instrument in the patient's body through entry openings as small as possible in size and/or to facilitate advancement of the instrument to the surgical site with as little trauma as possible to the patient. Another design advantage in rotary tissue cutting instruments is to maximize the internal diameter of the inner tubular member so that aspiration of tissue and/or fluid through the inner member can be accomplished with greater efficiency and with less risk of clogging. Yet a further design advantage in rotary tissue cutting instruments is to maintain an appropriate annular clearance between the internal diameter of the outer tubular member and the external diameter of the inner member to avoid jamming of the instrument and/or to provide efficient flow of irrigation fluid between the outer and inner members. In angled rotary tissue cutting instruments, it would also be a design advantage to minimize the number of structural components or parts required for the flexible region of the inner member, thereby reducing manufacturing and material costs, as well as reducing the risk of operational problems arising from structural complexity and/or multiple structural components. The foregoing design advantages must necessarily be balanced against the need to maintain sufficient strength and rigidity in the flexible inner members of angled rotary tissue cutting instruments when transmitting torque via the flexible regions, particularly considering that angled rotary tissue cutting instruments must normally be designed to operate at high rotational speeds and to withstand the forces imposed when cutting very hard or tenacious anatomical tissue.

Various designs previously proposed for the flexible regions in the inner members of angled rotary tissue cutting instruments have limited the extent to which the aforementioned design advantages can be optimized in angled rotary tissue cutting instruments. Some of the deficiencies associated with prior designs proposed for the flexible regions in the inner members of angled rotary tissue cutting instruments include increased radial thickness of the annular wall of the inner tubular member along the flexible region resulting in a larger external diameter and/or smaller internal diameter for the inner member, the need for multiple assembled structural parts to form the flexible region, constriction of the internal diameter of the flexible region when transmitting torque within an angled region of the outer tubular member, longitudinal stretching of the flexible region, and insufficient strength and rigidity in the flexible region limiting the range of bend angles over which the flexible region is able to effectively transmit torque. Designs for the flexible regions of inner tubular members of angled rotary tissue cutting instruments that result in the inner tubular member being of larger external diametric size normally require that the angled outer tubular member be of larger external diametric size in order to rotatably receive the inner member while maintaining the appropriate annular clearance between the outer and inner members. Designs for the flexible regions of inner tubular members of angled rotary tissue cutting instruments that result in the inner tubular member having a smaller internal diameter or that result in constriction of the internal diameter will typically have a negative impact on the ability to aspirate tissue and/or fluid through the inner tubular member. Designs for the flexible regions of inner tubular members of angled rotary tissue cutting instruments that require multiple assembled structural parts generally result in the inner tubular member being of higher cost and at increased risk of operational problems. Operational problems are also more likely to occur in inner tubular members of angled rotary tissue cutting instruments in which the design for the flexible region in the inner member makes the flexible region prone to longitudinal stretching.

In some flexible inner tubular members of angled rotary tissue cutting instruments, the flexible regions are formed of a plurality of concentric spirals, typically an inner spiral, a middle spiral and an outer spiral attached to one another at their ends. Each spiral is formed by winding a flat strip of material, with alternating spirals being wound in opposite rotational directions about a central longitudinal axis of the inner member as represented by U.S. Pat. No. 4,646,738 to Trott, U.S. Pat. No. 5,286,253 to Fucci and U.S. Pat. No. 5,540,708 to Lim et al. It has also been proposed to provide shafts having flexible regions made up of concentric coils of wound wire of circular cross-section, rather than wound flat strips of material, as represented by U.S. Pat. No. 5,437,630 to Daniel et al and U.S. Pat. No. 5,529,580 to Kusunoki et al and by German Patent DE 3828478 Al. The radial dimension or thickness of the annular wall of a flexible region comprised of multiple concentric spirals or coils tends to be substantial since it includes the individual thickness of each spiral or coil. Flexible regions of this type tend to result in flexible inner tubular members of larger external diametric sizes requiring diametrically larger outer tubular members, and/or of smaller internal diameters leading to reduced aspiration capability. In addition, flexible inner tubular members having these types of flexible regions will ordinarily be associated with higher material costs due to the multiple structural components involved and with higher manufacturing costs associated with producing and assembling the different structural components. The risk of operational problems may be greater due to the presence of multiple structural components and increased structural complexity, and the securement or attachment sites for the multiple spirals or coils present the potential for structural failure.

Another design approach for the flexible regions in the flexible inner tubular members of angled rotary tissue cutting instruments involves a single continuous spiral or helical cut formed in an inner tube, and one or more layers of spiral wrap disposed over the cut region of the inner tube as represented by U.S. Pat. No. 6,533,749 B1 to Mitusina et al and U.S. Pat. No. 6,656,195 B2 to Peters et al, and by United States Patent Application Publication No. US2005/0090849 A1 to Adams. The one or more layers of spiral wrap are each formed by winding a flat strip of material over the cut region in the inner tube and attaching the ends of the strip to the tube. The helical cut and the one or more layers of spiral wrap are arranged so that their rotational direction or turn about a central longitudinal axis of the inner member alternate in opposite directions. The Peters et al patent discloses the helical cut in the inner tube as having a dovetail pattern. The extent to which it is possible to minimize the radial dimension or thickness of the annular wall of a flexible region comprised of an inner tube and one or more layers of spiral wrap over a cut region of the tube is limited by the fact that the wall thickness of the inner tube and the thickness of each layer of spiral wrap contribute cumulatively to the radial dimension of the annular wall formed by the flexible region. Furthermore, the inner tube and each spiral wrap are separate structural components assembled during manufacture, giving rise to issues of increased cost and structural complexity.

U.S. Pat. No. 7,338,495 B2 to Adams is an example of a flexible region in a flexible inner tubular member of an angled rotary surgical cutting instrument formed of a helical cut in an inner tube, a layer of adhesive disposed over the cut region of the inner tube, and a heat shrunk sleeve disposed over the cut region of the inner tube and being bonded thereto by the adhesive. The helical cut is formed in the inner tube in a stepped pattern. Again, the radial thickness of the annular wall formed by the flexible region is made up of the individual thicknesses of the inner tube wall, the adhesive layer, and the wall of the sleeve. The flexible region requires multiple parts or materials in addition to the inner tube, and is still somewhat complicated from a manufacturing standpoint.

Flexible regions have also been provided in the inner tubular members of angled rotary tissue cutting instruments by forming disconnected slots or openings in an inner tube as illustrated by U.S. Pat. No. 5,152,744, U.S. Pat. No. 5,322,505 and U.S. Pat. No. 5,510,070 to Krause et al, U.S. Pat. No. 5,620,415 to Lucey et al, and U.S. Pat. No. 5,620,447 to Smith et al. Each slot is filled with a pliable material in a multi-step process carried out after the slots are formed. The preferred slot configuration described in the Krause et al, Lucey et al and Smith et al patents involves circumferentially discontinuous slots disposed in parallel spaced relation, the slots being arranged perpendicular to the longitudinal axis of the inner tube.

U.S. Pat. No. 6,053,922 to Krause et al pertains to a flexible shaft for reaming the medullary space in bones. In contrast to the flexible inner members of angled rotary tissue cutting instruments, the flexible shaft of Krause et al '922 is not designed to be rotatably disposed within a rigid outer tubular member, and is thusly not subject to the same design considerations as the inner members of rotary tissue cutting instruments and of angled rotary tissue cutting instruments in particular. In further distinction to the flexible inner tubular members of angled rotary tissue cutting instruments, the flexible shaft of Krause et al '922 is said to be an elongated tubular member of substantial wall thickness. A flexible inner tubular member of substantial wall thickness would be undesirable in an angled rotary tissue cutting instrument because it would result in a reduction in the internal diameter of the inner member, which would reduce aspiration capability, and/or it would require an outer member of larger external diameter to accommodate the inner member, which would require larger size entry openings in the patient's body for introduction of the instrument. The tubular member of Krause et al '922 comprises a slot, said to be of substantial width, extending spirally around the tubular member but in an undulating or serpentine pattern that deviates from a consistently spiral pattern. The undulations in the different slot patterns disclosed by Krause et al '922 are important in order to form interlocking regions in the tubular member that Krause et al '922 relies on to transmit torque.

Despite the numerous different design approaches previously proposed for the flexible inner members of angled rotary tissue cutting instruments, it was not recognized until the present invention that a flexible region comprising a plurality of intertwined helical cuts, each following a consistently helical path, formed in a tubular body of the inner member would provide numerous design advantages, including the advantages of eliminating the need for the flexible region to include an additional structure or layer of material over the helically cut region of the tubular body or within the cuts themselves, increased rigidity and torsional strength, resistance to stretching in the longitudinal axial direction of the inner member, preservation of the integrity of the internal diameter of the inner member, and the capability to transmit torque within angled outer tubular members having a broad range of bend angles.

### SUMMARY OF THE INVENTION

The present invention is generally characterized in a flexible inner member for being rotatably disposed within an angled outer tubular member of a rotary tissue cutting instrument. The outer tubular member includes a distal end, a longitudinal internal passage, an open proximal end communicating with the passage, an angled region between the distal and proximal ends, and an opening in the distal end communicating with the internal passage. The flexible inner member has a distal end, a proximal end, a tubular body between the distal and proximal ends of the inner member, a cutting element at the distal end of the inner member, and a flexible region for being disposed within the angled region of the outer tubular member. When the inner member is rotatably disposed within the internal passage of the outer tubular member, the cutting element is exposed from the opening in the outer tubular member, and the flexible region is disposed within the angled region to transmit torque to rotate the cutting element while conforming to the configuration of the angled region. The tubular body of the inner member has a central longitudinal or helix axis and a cylindrical wall having a wall thickness between external and internal diameter surfaces of the cylindrical wall. The flexible region is formed of a plurality of intertwined helical cuts in the tubular body, each helical cut extending entirely through the wall thickness of the cylindrical wall and extending longitudinally along the body in a consistently helical path about the helix axis. The helical cuts are congruent but are translated or longitudinally offset from one another in a direction parallel to the helix axis. The helical cuts result in formation of a plurality of intertwined helical strip members in the cylindrical wall of the tubular body. Each helical strip member has opposed ends unified integrally and unitarily or monolithically with the cylindrical wall. The helical strip members have the same pitch and helix angle and are of equal width. Illustrative embodiments involve two intertwined helical cuts forming two intertwined helical strip members, three intertwined helical cuts forming three intertwined helical strip members, and four intertwined helical cuts forming four intertwined helical strip members.

### BRIEF DESCRIPTION OF THE DRAWINGS

FIG. 1 is an exploded side view of an angled rotary tissue cutting instrument.

FIG. 2A is a broken side view of a flexible region of a flexible inner member of the angled rotary tissue cutting instrument.

FIG. 2B is a broken perspective view of the flexible region of FIG. 2A.

FIG. 3A is a broken side view of an alternative flexible region of a flexible inner member of the angled rotary tissue cutting instrument.

FIG. 3B is a broken perspective view of the flexible region of FIG. 3A.

FIG. 4A is a broken side view of another alternative flexible region of a flexible inner member of the angled rotary tissue cutting instrument.

FIG. 4B is a broken perspective view of the flexible region of FIG. 4A.

### DETAILED DESCRIPTION OF THE INVENTION

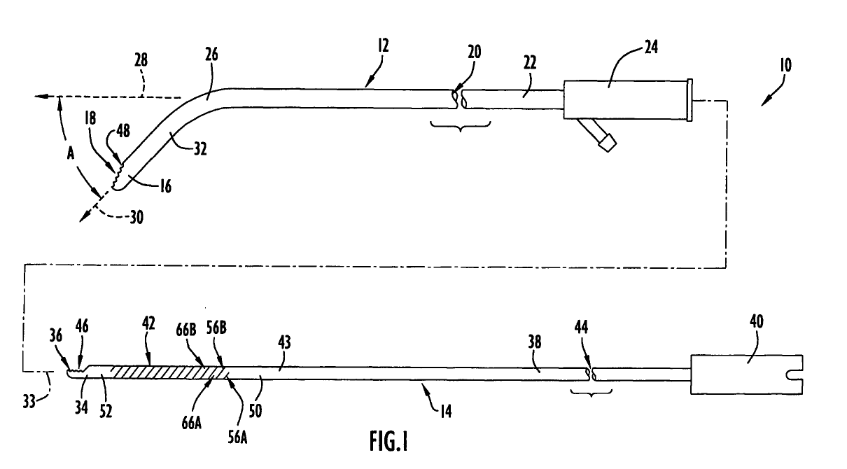

An angled rotary tissue cutting instrument 10 is depicted in FIG. 1 and comprises an elongate angled outer tubular member 12 and an elongate flexible inner member 14 for being rotatably disposed within the outer tubular member 12. The outer tubular member 12 has a distal end 16 with an opening 18 therein in communication with the internal passage 20 in the outer tubular member. The outer tubular member 12 has a proximal length portion 22 terminating at an open proximal end typically secured in an outer member hub 24 designed for engagement with a powered surgical handpiece (not shown) in a conventional manner. The outer tubular member 12 is provided with one or more angled, curved or bent regions 26 along the length thereof, such that the outer tubular member 12 has an angled configuration. Each angled region 26 in the outer tubular member 12 defines a bend angle A corresponding to the angle defined between length portions of the outer tubular member 12 that are joined by the angled region. The outer tubular member 12, for example, has a bend angle A defined between the central longitudinal axis 28 of the proximal length portion 22 of the outer member 12 and a central longitudinal axis 30 of a distal length portion 32 of the outer member 12 which is joined to the proximal length portion 22 by the angled region 26. The size and the direction of the bend angle A can vary individually for each angled region 26. The outer tubular member 12 illustrated in FIG. 1 has one angled region 26 with a bend angle A extending in a downward direction from proximal length portion 22.

As a result of its angled configuration, the outer tubular member 12 is not longitudinally straight along its length. However, the outer tubular member 12 can initially be provided in a longitudinally straight configuration, without the one or more angled regions 26, and can be bent from the longitudinally straight configuration in any suitable manner to obtain the angled configuration desired for the outer tubular member. Accordingly, bending the outer tubular member 12 from the longitudinally straight configuration to the desired angled configuration will involve bending the outer tubular member 12 as needed to obtain the desired number of angled regions 26 at the desired location or locations along the length of the outer tubular member and extending in the desired direction or directions at the desired bend angle or angles A. It should be appreciated that the outer tubular member 12 can be bent from the longitudinally straight configuration to the angled configuration with or without the inner member 14 disposed within the outer tubular member 12. The outer tubular member 12 is rigid in a longitudinally straight configuration but is able to be bent to form the desired angled region(s) when sufficient bending force is applied. The outer tubular member 12 is or remains rigid after bending to form the one or more angled regions.

The inner member 14 has a central longitudinal axis 33, a distal end 34 provided with or formed as a cutting element 36, a proximal length portion 38 terminating at a proximal end that is typically secured in an inner member hub 40, and one or more flexible regions 42 between the cutting element 36 and the inner member hub 40. The one or more flexible regions 42 impart flexibility to the inner member that allows the inner member to bend along its central longitudinal axis 33. When the inner member 14 is assembled with the outer tubular member 12 to cut anatomical tissue, the inner member 14 will extend through the outer member hub 24 and will be rotatably disposed within the internal passage 20 of the outer tubular member 12 with the cutting element 36 exposed from the opening 18 in the outer member, with the one or more flexible regions 42 disposed within the one or more angled regions 26 of the outer member, and with the inner member hub 40 disposed proximally of the outer member hub 24 for engagement with the powered surgical handpiece. The powered surgical handpiece is operated in a conventional manner to rotate the inner member 14 relative to and within the outer tubular member 12, and the one or more flexible regions 42 transmit torque to rotate the cutting element 36 while conforming to the angled configuration of the outer tubular member 12. As the inner member 14 is rotated within the outer tubular member 12, the cutting element 36 exposed from the opening 18 will cut anatomical tissue contacted with the cutting element 36.

The inner member 14 comprises a cylindrical tubular body 43 coaxial with the central longitudinal axis 33 and having an internal lumen 44 extending longitudinally within the tubular body. The tubular body 43 preferably has an open end forming the proximal end of the inner member 14 and preferably extends from the proximal end of the inner member 14 to the cutting element 36, which is the case for tubular body 43. Preferably, the tubular body 43 is an integral and unitary or monolithic tube from the proximal end of the inner member 14 to the cutting element 36, which is also the case for tubular body 43. Accordingly, the flexible inner member 14 is a flexible inner tubular member. As described further below, the one or more flexible regions 42 are each formed by a plurality of intertwined helical cuts in the tubular body 43.

The cutting element 36 can have various cutting configurations effective to cut anatomical tissue including the various cutting configurations conventionally used for the inner members of rotary tissue cutting instruments. The cutting element 36 can be a structure that is hollow or provided with an interior cavity or channel in communication with the lumen 44 of the tubular body 43. The cutting element 36 can be a structure formed separate from and attached to the tubular body 43. The distal end 34 of the inner tubular member 14 can have an opening 46 therein in communication with the internal lumen 44 of the inner member 14, and the opening 46 can communicate with the lumen 44 via the interior cavity or channel in the structure that forms the cutting element 36. The cutting configuration for the cutting element 36 can include one or more cutting surfaces or edges along the periphery of the opening 46 as is the case for the cutting element 36 of the inner member 14 depicted in FIG. 1. The cutting surfaces or edges of the cutting element 36 can be defined by cutting tooth formations, as is also the case for the cutting element 36 of inner member 14. The cutting surfaces or edges of the cutting element 36 can be defined by flute formations as in a bur tip, for example.

The distal end 16 of the outer tubular member 12 can be provided with or formed as a cutting element 48 that cooperates with the cutting element 36 of the inner member 14 to cut anatomical tissue. The cutting element 48 can have various cutting configurations effective to cut anatomical tissue in cooperation with the cutting element 36, and the various cutting configurations conventionally used for the outer members in rotary tissue cutting instruments can be used for the cutting configuration of the cutting element 48. The cutting configuration for the cutting element 48 can include one or more cutting surfaces or edges along the periphery of the opening 18 as is the case for the cutting element 48 of the outer tubular member 12 depicted in FIG. 1. The cutting surfaces or edges of the cutting element 48 can be defined by cutting tooth formations, as is also the case for the cutting element 48. Typically, the cutting elements 36 and 48 cooperate to cut anatomical tissue as a result of rotation of the one or more cutting surfaces or edges of the cutting element 36 past the one or more cutting surfaces or edges of the cutting element 48.

As the cutting element 36 is rotated, the opening 46 in the inner member 14 will come into alignment with the opening 18 in the outer tubular member 12, allowing anatomical tissue and/or fluid to enter the lumen 44 of the inner member 14 through the aligned openings 18 and 46. Through the application of vacuum or suction to the lumen 44, typically via a connection at a proximal end of the instrument 10 in a conventional manner, the lumen 44 can serve as an aspiration passage by which suction is applied at the surgical site via the aligned openings 18 and 46 and by which anatomical tissue and/or fluid is/are drawn into the lumen 44 through the aligned openings 18 and 46 for evacuation through the instrument 10.

In order for the inner member 14 to rotate within the outer tubular member 12 without jamming, an appropriate annular clearance or gap is present between the internal diameter of the outer tubular member 12 and the external diameter of the inner member 14 when the members 12 and 14 are assembled to cut tissue. The annular clearance or gap between the outer and inner members 12 and 14 can serve as an irrigation passage by which irrigation fluid supplied to the annular clearance, typically from a proximal end of the instrument 10, is conveyed distally and released at the surgical site through the opening 18 in the outer tubular member 12.

The inner member 14 can have a single flexible region 42 of sufficient length to reside in and conform to the configuration of one or more angled regions 26 in the outer tubular member 12. The inner member 14 can have a plurality of flexible regions 42, each of sufficient length to reside in and conform to the configuration of a corresponding angled region 26 in the outer tubular member 12. Each flexible region 42 can be disposed adjacent and/or between rigid or non-flexible length segments of the tubular body 43. The inner member 14 is an example of one having a single flexible region 42 disposed between rigid or non-flexible length segments 50 and 52 of the tubular body 43, the single flexible region 42 being located along the length of the inner member 14 to reside within the single angled region 26 in the outer tubular member 12 and being of sufficient length to conform to the configuration of the single angled region 26 when the inner member 14 is assembled with the outer member 12 to cut anatomical tissue. The length segment 50 of the tubular body 43 is part of the proximal length portion 38, which will be disposed within the proximal length portion 22 of the outer member 12 when the inner member is assembled with the outer member to cut anatomical tissue. The length segment 52 of the tubular body 43 may thusly be considered a distal length portion and will be disposed within the distal length portion 32 of the outer member 12 when the inner and outer members are assembled to cut anatomical tissue.

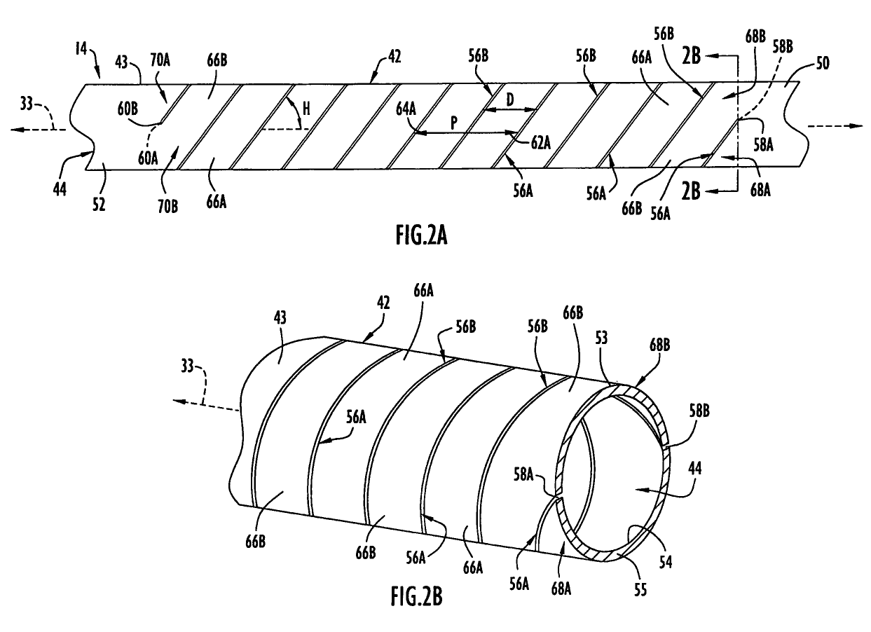

The flexible region 42, which is best depicted in FIGS. 1, 2A and 2B, comprises a plurality of intertwined helical cuts 56A and 56B formed in the tubular body 43 of the inner member 14. As best shown in FIG. 2B, the tubular body 43 has a wall with an external diameter surface 53, an internal diameter surface 54 defining the lumen 44, and a radial wall thickness 55 between the external and internal diameter surfaces. Prior to the intertwined helical cuts 56A and 56B being formed therein, the wall of the tubular body 43 along the flexible region 42 and along the length segments 50 and 52 is a rigid, solid and continuous, cylindrical, annular wall coaxial with the central longitudinal axis 33. The flexible region 42 is an example of one comprising two intertwined helical cuts 56A and 56B, each extending through the entire wall thickness 55 of the cylindrical wall of the tubular body 43 as best seen in FIG. 2B. The helical cut 56A has a starting point 58A on the tubular body 43 and has an end point 60A on the tubular body 43. The helical cut 56A is continuous from the starting point 58A to the end point 60A. The helical cut 56A rotates or turns about a helix axis, i.e. the central longitudinal axis 33 of tubular body 43, in a constant rotational direction between its starting point 58A and its end point 60A. The helical cut 56A follows a consistently helical path between its starting point 58A and its end point 60A on the tubular body 43. A consistently helical path is one that rotates or turns in one rotational direction about the helix axis, while moving parallel to the helix axis. Since the wall of the tubular body 43 is cylindrical, the helical path for the helical cut 56A is a circular helix. In addition, a consistently helical path is one in which for any two points along the helical path that are spaced from one another by a single, complete revolution, i.e. 360°, about the helix axis, for example intermediate points 62A and 64A in FIGS. 2A and 2B, the two points are spaced longitudinally from one another in a direction parallel with the central longitudinal or helix axis 33 by the pitch P of the helix. The pitch P may also be described as the width of one complete helix turn, measured along the helix axis. The helix defined by the helical path of the helical cut 56A has a helix angle H, which is the angle between the helix and the central longitudinal or helix axis 33. Another characteristic of a consistently helical path is that all points along the helical path between two points, such as intermediate points 62A and 64A, that demarcate a single complete revolution of the helix are at angle H to the helix axis. The flexible region 42 is an example of one where the helical cut 56A is a left-handed helical cut, rotating in a counterclockwise rotational direction about the central longitudinal or helix axis 33 of the tubular body 43 from the starting point 58A to the end point 60A and thusly having a left-hand turn. The helical cut 56A could, however, rotate clockwise about the axis 33 from the starting point 58A with a right-hand turn.

The helical cut 56B is similar to the helical cut 56A and has the same consistently helical path as the helical cut 56A but the helices defined by the helical cuts 56A and 56B differ by a translation along the central longitudinal or helix axis 33. The helical cut 56B has a starting point 58B on the tubular body 43 that is rotationally offset from the starting point 58A of helical cut 56A. As seen in FIGS. 2A and 2B, the starting point 58B is rotationally offset 180° from the starting point 58A about the central longitudinal or helix axis 33. Accordingly, starting points 58A and 58B are at 180° spaced radial locations about the central longitudinal or helix axis 33 of the tubular body 43. Similarly, the end point 60B of the helical cut 56B is rotationally offset 180° from the end point 60A of the helical cut 56A about the central longitudinal or helix axis 33. The end point 60A is depicted in FIG. 2A as being in line with the starting point 58B in the direction parallel to the central longitudinal axis 33, which places the end point 60B in line with the starting point 58A in the direction parallel to axis 33. The longitudinal distance between the starting points 58A, 58B and the end points 60A, 60B and, therefore, the lengths of the helical cuts 56A, 56B, will depend on the length required for the flexible region 42. In the case of helical cuts 56A and 56B, the end points 60A, 60B are located on the tubular body 43 distally of the starting points 58A, 58B. It should be appreciated, however, that the starting and end points can be reversed, and that the points 60A, 60B can be the starting points for the helical cuts 56A, 56B while the points 58A, 58B can be the end points for the helical cuts 56A, 56B.

The helical cuts 56A and 56B are congruent, with the helical cut 56B rotating or turning about the central longitudinal or helix axis 33 in the same rotational direction as the helical cut 56A and with the same pitch P and helix angle H as the helical cut 56A. However, the helical cut 56B is translated or longitudinally offset from the helical cut 56A in a direction parallel to the central longitudinal or helix axis 33 by an offset distance D, which is equal to one-half the pitch P. Accordingly, the helical cuts 56A and 56B are intertwined and result in the formation of two intertwined helical strip members 66A and 66B in the cylindrical wall of the tubular body 43. Helical strip member 66A is formed of a portion of the cylindrical wall of the tubular body 43 between the helical cuts 56A and 56B, and helical strip member 66B is formed of the remaining portion of the cylindrical wall between the helical cuts 56A and 56B. The helical strip member 66A has a first side edge formed by helical cut 56B, a second side edge formed by helical cut 56A, and a width between its side edges. The helical strip member 66B has its first side edge formed by helical cut 56A, has its second side edge formed by helical cut 56B, and has a width between its side edges that is the same as the width of helical strip member 66A. As seen in FIGS. 2A and 2B, the helical strip member 66A has a first or starting end 68A that extends arcuately from the starting point 58A to the starting point 58B in the direction of rotation of the helical strip member 66A about the central longitudinal or helix axis 33 (looking distally along the axis 33 toward the end points 60A, 60B), which is the same as the rotational direction of the helical cuts 56A, 56B, i.e. counterclockwise. The helical strip member 66B has a first or starting end 68B that extends arcuately from the starting point 58B to the starting point 58A in the direction of rotation of the helical strip member 66B about the central longitudinal or helix axis 33 (again looking distally along the axis 33 toward the end points 60A, 60B), which is the same as the direction of rotation of the helical strip member 66A. The first ends 68A and 68B of the helical strip members 66A and 66B are unified integrally and unitarily or monolithically with the solid, rigid, uncut portion 50 of the cylindrical wall of the tubular body 43, and both helical strip members 66A and 66B are thusly united to each other at their first ends 68A and 68B.

As seen in FIG. 2A, the helical strip members 66A and 66B have respective second or terminal ends 70A and 70B, opposite the first ends 68A and 68B. The second end 70A extends arcuately from the end point 60A to the end point 60B in the direction of rotation of the helical strip member 66A (looking distally along the axis 33). The second end 70B spans or extends rotationally about the axis 33 from the end point 60B to the end point 60A in the direction of rotation of the helical strip member 66B (again looking distally along the axis 33). The second ends 70A and 70B of the helical strip members 66A and 66B are unified integrally and unitarily or monolithically with the solid, rigid, uncut portion 52 of the cylindrical wall of the tubular body 43, and both helical strip members 66A and 66B are therefore united to each other at their second ends 70A and 70B. The radial dimension or thickness of the annular wall of the inner tubular member 14 along the flexible region 42 is the same as the wall thickness 55 of the tubular body 43 along the helically cut region of the body 43, which is the same as the wall thickness along the solid, rigid, uncut portions 50, 52 of the cylindrical wall of the body 43. The helical cuts 56A and 56B can be formed in the tubular body 43 in any suitable manner, preferably by laser cutting.

The flexible region 42 has numerous advantages including but not limited to increased rigidity and torsional strength, greater resistance to stretching in the longitudinal direction of the inner tubular member 14, and preservation of the integrity of the internal diameter of the tubular body 43 when transmitting torque during rotation of the inner tubular member 14 within the angled outer tubular member 12. The flexible region 42 has the further advantage of not requiring any additional structural component(s) and/or material(s) over or within the helical cuts in order to operate effectively as the flexible region for an inner member of an angled rotary tissue cutting instrument. An additional advantage is that the annular wall thickness of the inner member 14 along the flexible region 42 can be better minimized in order to better minimize the external diameter of the inner member and/or to better maximize the internal diameter of the inner member. Also, eliminating the need for additional structural components and/or materials presents the advantage of allowing the flexible region 42 to be produced at lower cost and with greater structural simplicity for a reduced risk of operational problems. Although the flexible region 42 does not require any additional structural component(s) over the helical cuts, it is possible to provide a very thin-walled sleeve or sheath over the helically cut region of the tubular body while retaining the aforementioned advantages. The flexible region 42 is an example of one in which the helical cuts 56A and 56B have a width of or about 0.062 inch and is especially well-suited for use with an angled outer tubular member 12 having a bend angle A of up to 30°. By forming the helical cuts 56A and 56B to have a width of or about 0.050 inch, the flexible region 42 is particularly well-suited for use with an angled outer tubular member 12 having a bend angle A of up to 45°.

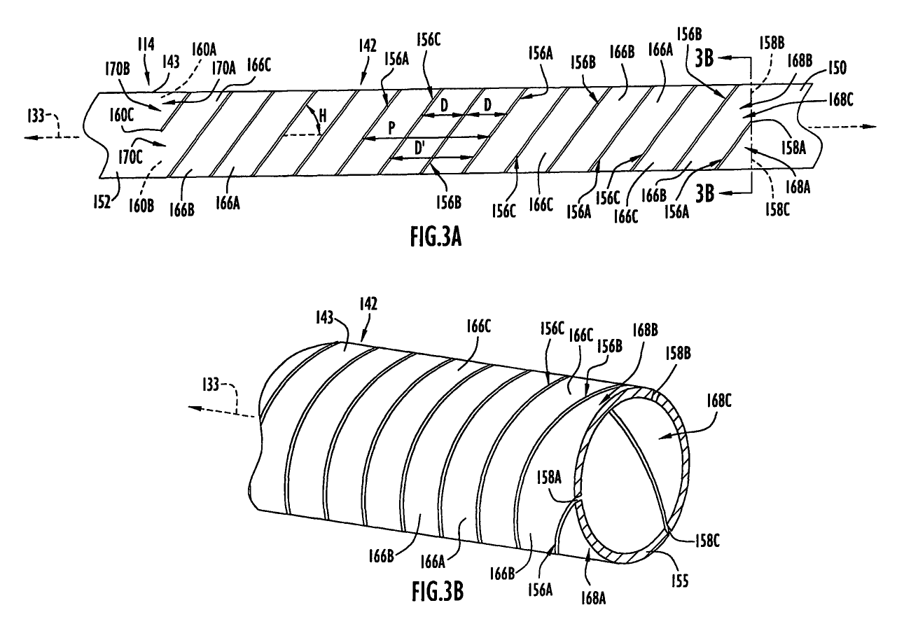

FIGS. 3A and 3B illustrate an alternative flexible region 142 for an inner member 114 of the angled rotary tissue cutting instrument. The flexible region 142 is similar to flexible region 42 but comprises three intertwined helical cuts 156A, 156B and 156C in the tubular body 143 of the inner member 114. The helical cuts 156A, 156B and 156C are similar to helical cuts 56A and 56B, and they extend continuously in a consistently helical path between their respective starting points 158A, 158B and 158C and their respective end points 160A, 160B and 160C on the tubular body 143. The starting points 158A, 158B and 158C are rotationally offset from one another about the central longitudinal or helix axis 133 of the tubular body 143. In particular, the starting points 158A, 158B and 158C are disposed at equally spaced radial locations about the central longitudinal or helix axis 133 of the tubular body 143. Accordingly, the starting point 158B for helical cut 156B is radially spaced 120° from the starting point 158A for helical cut 156A, and the starting point 158C for helical cut 156C is radially spaced 120° from the starting points 158A and 158B. The helical cuts 156A, 156B and 156C rotate or turn in the same rotational direction about the central longitudinal or helix axis 133 of tubular body 143 and have the same pitch P and helix angle H. The flexible region 142 is representative of one in which the helical cuts 156A, 156B and 156C rotate or turn in a counterclockwise rotational direction about the central longitudinal or helix axis 133 of the tubular body 143 from their starting points to their end points and thusly have a left-hand turn. The helical cuts 156A, 156B and 156C could, however, rotate clockwise with a right-hand turn.

The helical cuts 156A, 156B and 156C are congruent but differ by a translation or longitudinal offset in a direction parallel to the central longitudinal or helix axis 133. The helical cut 156B is translated or longitudinally offset from the helical cut 156A by an offset distance D equal to one-third the pitch P. The helical cut 156C is translated or longitudinally offset from the helical cut 156B by the offset distance D and from the helical cut 156A by an offset distance D′ equal to two-thirds the pitch P. The helical cuts 156A, 156B and 156C are intertwined and result in the formation of three intertwined helical strip members 166A, 166B and 166C in the cylindrical wall of the tubular body 143. Helical strip member 166A is formed of the portion of the cylindrical wall of the tubular body 143 between the helical cuts 156A and 156C. Helical strip member 166B is formed of the portion of the cylindrical wall of the tubular body 143 between the helical cuts 156A and 156B. Helical strip member 166C is formed of the portion of the cylindrical wall of the tubular body 143 between helical cuts 156B and 156C. The helical strip member 166A has its first side edge formed by helical cut 156C and its second side edge formed by helical cut 156A. The helical strip member 166B has its first side edge formed by helical cut 156A and its second side edge formed by helical cut 156B. The helical strip member 166C has its first side edge formed by helical cut 156B and its second side edge formed by helical cut 156C. The width of the helical strip members 166A, 166B and 166C between their side edges is the same for each helical strip member 166A, 166B and 166C. The first or starting end 168A for helical strip member 166A extends arcuately from the starting point 158A to the starting point 158C in the direction of rotation of the helical strip member 166A about the central longitudinal or helix axis 133 (looking distally along the axis 133 toward the end points 160A, 160B, 160C), which is the same as the rotational direction of the helical cuts 156A, 156B, 156C, i.e. counterclockwise. The first or starting end 168B for helical strip member 166B extends arcuately from the starting point 158B to the starting point 158A in the direction of rotation of the helical strip member 166B about the central longitudinal or helix axis 133 (again looking distally along the axis 133), which is the same as the direction of rotation of the helical strip member 166A. The first or starting end 168C for helical strip member 166C extends arcuately from the starting point 158C to the starting point 158B in the direction of rotation of the helical strip member 166C about the axis 133 (again looking distally along the axis 133), which is the same as the direction of rotation of the helical strip members 166A and 166B. The first ends 168A, 168B and 168C of the helical strip members 166A, 166B and 166C are unified integrally and unitarily or monolithically with the solid, rigid, uncut portion 150 of the cylindrical wall of the tubular body 143, and all three helical strip members 166A, 166B and 166C are thusly united to each other at their first ends 168A, 168B and 168C.

The end points 160A, 160B and 160C of the helical cuts 156A, 156B and 156C are located on the tubular body 143 at 120° spaced radial locations about the axis 133. The second or terminal end 170A of the helical strip member 166A extends arcuately from the end point 160A to the end point 160C in the direction of rotation of the helical strip member 166A about the axis 133 (looking distally along the axis 133). The second or terminal end 170B of helical strip member 166B extends arcuately from the end point 160B to the end point 160A in the direction of rotation of the helical strip member 166B about the axis 133 (again looking distally along axis 133). The second or terminal end 170C of helical strip member 166C extends arcuately from the end point 160C to the end point 160B in the direction of rotation of the helical strip member 166C about the axis 133 (again looking distally along axis 133). The second ends 170A, 170B and 170C are unified integrally and unitarily or monolithically with the solid, rigid, uncut portion 152 of the cylindrical wall of the tubular body 143, and all three helical strip members 166A, 166B and 166C are thusly united to each other at their second ends 170A, 170B and 170C. The radial dimension or thickness of the annular wall of the inner tubular member 114 along the flexible region 142 is the same as the wall thickness 155 of the tubular body 143 along the helically cut region of the body 143, which is the same as the wall thickness along the solid, rigid, uncut portions 150, 152 of the body 143. The flexible region 142 achieves the same advantages as those described above for the flexible region 42. The flexible region 142 is an example of one in which the helical cuts 156A, 156B and 156C have a width of or about 0.035 inch and is especially well-suited for use with an angled outer tubular member having a bend angle A of up to 70°.

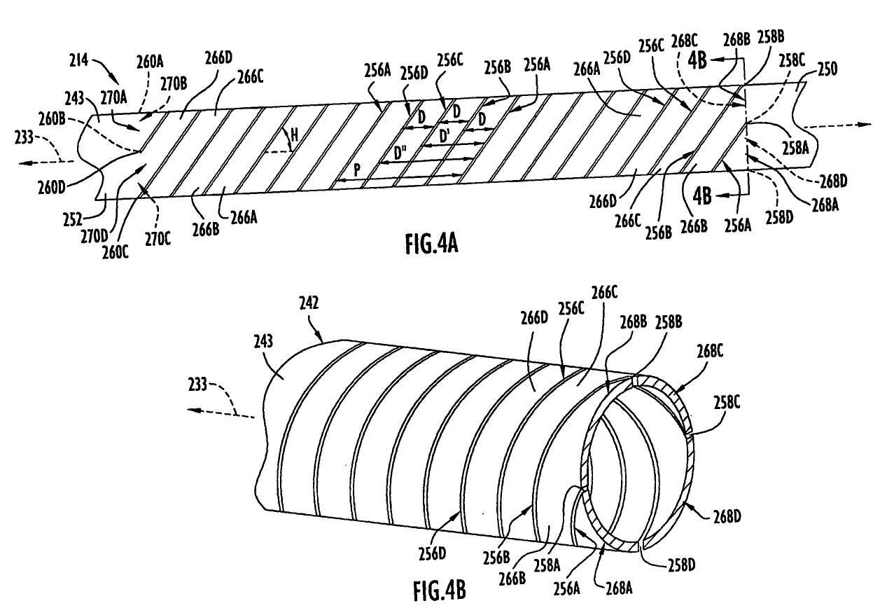

A further alternative flexible region 242 for an inner member 214 of the angled rotary tissue cutting instrument is shown in FIGS. 4A and 4B. The flexible region 242 is similar to flexible regions 42 and 142 but comprises four intertwined helical cuts 256A, 256B, 256C and 256D in the tubular body 243 of the inner member 214. The helical cuts 256A, 256B, 256C and 256D are similar to helical cuts 56A, 56B, 156A, 156B and 156C, and they extend continuously in a consistently helical path between their respective starting points 258A, 258B, 258C and 258D and their respective end points 260A, 260B, 260C and 260D on the tubular body 243. The starting points 258A, 258B, 258C and 258D are rotationally offset from one another about the central longitudinal or helix axis 233 of the tubular body 243. The starting points 258A, 258B, 258C and 258D are disposed at equally spaced radial locations about the central longitudinal or helix axis 233 of the tubular body 243. The starting point 258B for helical cut 256B is thusly radially spaced 90° from the starting point 258A for helical cut 256A. The starting point 258C for the helical cut 256C is radially spaced 90° from the starting point 258B and 180° from the starting point 258A. The starting point 258D for the helical cut 256D is radially spaced 90° from the starting points 258C and 258A, and 180° from the starting point 258B. The helical cuts 256A, 256B, 256C and 256D rotate or turn about the central longitudinal or helix axis 233 of the tubular body 243 in the same rotational direction and have the same pitch P and helix angle H. The flexible region 242 is representative of one in which the helical cuts 256A, 256B, 256C and 256D rotate or turn in a counterclockwise rotational direction about the central longitudinal or helix axis 233 of the tubular body 243 from their starting points to their end points. The helical cuts 256A, 256B, 256C and 256D therefore have a left-hand turn, but they could be made with a right-hand turn.

The helical cuts 256A, 256B, 256C and 256D are congruent but differ by a translation or longitudinal offset in a direction parallel to the central longitudinal or helix axis 233. The helical cut 256B is translated or longitudinally offset from the helical cut 256A by an offset distance D equal to one-fourth the pitch P. The helical cut 256C is translated or longitudinally offset from the helical cut 256B by the offset distance D and from the helical cut 256A by the offset distance D′ equal to one-half the pitch P. The helical cut 256D is translated or longitudinally offset from the helical cut 256C by the offset distance D, from the helical cut 256B by the offset distance D′, and from the helical cut 256A by an offset distance D″ that is three-fourths the pitch P.

The helical cuts 256A, 256B, 256C and 256D are intertwined and result in the formation of four intertwined helical strip members 266A, 266B, 266C and 266D in the cylindrical wall of the tubular body 243. Helical strip member 266A is formed of the portion of the cylindrical wall of the body 243 between the helical cuts 256A and 256D. Helical strip member 266B is formed of the portion of the cylindrical wall of body 243 between the helical cuts 256B and 256A. Helical strip member 266C is formed of the portion of the cylindrical wall of body 243 between helical cuts 256C and 256B. Helical strip member 266D is formed of the portion of the cylindrical wall of body 243 between helical cuts 256D and 256C. The first and second side edges of helical strip member 266A are formed, respectively, by helical cuts 256D and 256A. The first and second side edges of helical strip member 266B are formed, respectively, by helical cuts 256A and 256B. The first and second side edges of helical strip member 266C are formed, respectively, by helical cuts 256B and 256C. The first and second side edges of helical strip member 266D are formed, respectively, by helical cuts 256C and 256D. The width of the helical strip members 266A, 266B, 266C and 266D between their first and second side edges is the same for each helical strip member.

The first or starting end 268A for helical strip member 266A extends arcuately from the starting point 258A to the starting point 258D in the direction of rotation of the helical strip member 266A about the central longitudinal or helix axis 233 (looking distally along the axis 233 toward the end points 260A, 260B, 260C and 260D), which is the same as the rotational direction of the helical cuts 256A, 256B, 256C and 256D, i.e. counterclockwise. The first or starting end 268B for helical strip member 266B extends arcuately from the starting point 258B to the starting point 258A in the direction of rotation of the helical strip member 266B about the central longitudinal or helix axis 233 (again looking distally along the axis 233), which is the same as the direction of rotation of the helical strip member 266A. The first or starting end 268C for helical strip member 266C extends arcuately from the starting point 258C to the starting point 258B in the direction of rotation of the helical strip member 266C about the axis 233 (again looking distally along the axis 233), which is the same as the direction of rotation of the helical strip members 266A and 266B. The first or starting end 268D for helical strip member 266D extends arcuately from the starting point 258D to the starting point 258C in the direction of rotation of the helical strip member 266D about the axis 233 (again looking distally along the axis 233), which is the same as the direction of rotation of the helical strip members 266A, 266B and 266C. The first ends 268A, 268B, 268C and 268D of the helical strip members 266A, 266B, 266C and 266D are unified integrally and unitarily or monolithically with the solid, rigid, uncut portion 250 of the cylindrical wall of the tubular body 243, and all four helical strip members 266A, 266B, 266C and 266D are thusly united to each other at their first ends 268A, 268B, 268C and 268D.

The end points 260A, 260B, 260C and 260D of the helical cuts 256A, 256B, 256C and 256D are located on the tubular body 243 at 90° spaced radial locations about the axis 233. The second or terminal end 270A of the helical strip member 266A extends arcuately from the end point 260A to the end point 260D in the direction of rotation of the helical strip member 266A about the axis 233 (looking distally along the axis 233). The second or terminal end 270B of helical strip member 266B extends arcuately from the end point 260B to the end point 260A in the direction of rotation of the helical strip member 266B about the axis 233 (again looking distally along axis 233). The second or terminal end 270C of helical strip member 266C extends arcuately from the end point 260C to the end point 260B in the direction of rotation of the helical strip member 266C about the axis 233 (again looking distally along axis 233). The second or terminal end 270D of helical strip member 266D extends arcuately from the end point 260D to the end point 260C in the direction of rotation of the helical strip member 266D about the axis 233 (again looking distally along axis 233). The second ends 270A, 270B, 270C and 270D are unified integrally and unitarily or monolithically with the solid, rigid, uncut portion 252 of the cylindrical wall of the tubular body 243, and all four helical strip members 266A, 266B, 266C and 266D are thereby united to each other at their second ends 270A, 270B, 270C and 270D. The radial dimension or thickness of the annular wall of the inner tubular member 214 along the flexible region 242 is the same as the wall thickness 255 of the tubular body 243 along the helically cut region of the body 243, which is the same as the wall thickness along the solid, rigid, uncut portions 250, 252 of the body 243.

The flexible region 242 achieves the same advantages as described above for the flexible region 42. The flexible region 242 is an example of one in which the helical cuts 256A, 256B, 256C and 256D have a width of or about 0.025 inch and is especially well-suited for use with an angled outer tubular member having a bend angle A of up to 90°.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. An angled rotary tissue cutting instrument for cutting anatomical tissue, comprising
an elongate angled outer tubular member having a distal end, a longitudinal internal passage, an open proximal end communicating with said passage, an angled region between said distal end and said proximal end, and an opening in said distal end communicating with said passage; and
a flexible inner member for being rotatably disposed within said outer tubular member, said inner member having a distal end, a proximal end, a tubular body between said distal end of said inner member and said proximal end of said inner member, a cutting element at said distal end of said inner member, said cutting element being exposed from said opening to cut anatomical tissue when said inner member is rotatably disposed within said outer tubular member, and a flexible region for being disposed within said angled region to transmit torque to rotate said cutting element while conforming to the configuration of said angled region when said inner member is rotated within said outer tubular member, said tubular body having a central longitudinal axis and a cylindrical wall with an external diameter surface, an internal diameter surface, and a wall thickness between said external diameter surface and said internal diameter surface, said flexible region comprising a plurality of intertwined helical cuts in said tubular body, each of said helical cuts extending entirely through said wall thickness and extending longitudinally along said body in a consistently helical path about said axis, said helical paths of said cuts being congruent and being longitudinally offset from one other in a direction parallel to said axis.

2. The angled rotary tissue cutting instrument recited in claim 1 wherein each of said helical cuts has a starting point on said tubular body, an end point on said tubular body, and each extends continuously along said tubular body in said consistently helical path from said starting point to said end point.

3. The angled rotary tissue cutting instrument recited in claim 2 wherein said plurality of intertwined helical cuts comprises two intertwined helical cuts, said starting points of said helical cuts being located on said tubular body at 180° spaced radial locations about said axis, and said end points of said helical cuts being located on said tubular body at 180° spaced radial locations about said axis.

4. The angled rotary tissue cutting instrument recited in claim 3 wherein said helical cuts have the same pitch, have the same helix angle, and are longitudinally offset by a distance of one-half said pitch.

5. The angled rotary tissue cutting instrument recited in claim 4 wherein said helical cuts have a width of about 0.062 inch.

6. The angled rotary tissue cutting instrument recited in claim 5 wherein said angled region defines a bend angle in said outer tubular member and said flexible region conforms to a bend angle of up to 30°.

7. The angled rotary tissue cutting instrument recited in claim 3 wherein said helical cuts have a width of about 0.050 inch.

8. The angled rotary tissue cutting instrument recited in claim 7 wherein said angled region defines a bend angle in said outer tubular member and said flexible region conforms to a bend angle of up to 45°.

9. The angled rotary tissue cutting instrument recited in claim 2 wherein said plurality of intertwined helical cuts comprises three intertwined helical cuts, said starting points of said helical cuts being located on said tubular body at 120° spaced radial locations about said axis, and said end points of said helical cuts being located on said tubular body at 120° spaced radial locations about said axis.

10. The angled rotary tissue cutting instrument recited in claim 9 wherein said helical cuts have the same pitch, have the same helix angle, and are longitudinally offset by a distance of one-third said pitch.

11. The angled rotary tissue cutting instrument recited in claim 10 wherein said helical cuts have a width of about 0.035 inch.

12. The angled rotary tissue cutting instrument recited in claim 11 wherein said angled region defines a bend angle in said outer tubular member and said flexible region conforms to a bend angle of up to 70°.

13. The angled rotary tissue cutting instrument recited in claim 2 wherein said plurality of intertwined helical cuts comprises four intertwined helical cuts, said starting points of said helical cuts being located on said tubular body at 90° spaced radial locations about said axis, and said end points of said helical cuts being located on said tubular body at 90° spaced radial locations about said axis.

14. The angled rotary tissue cutting instrument recited in claim 13 wherein said helical cuts have the same pitch, have the same helix angle, and are longitudinally offset by a distance of one-fourth said pitch.

15. The angled rotary tissue cutting instrument recited in claim 14 wherein said helical cuts have a width of about 0.025 inch.

16. The angled rotary tissue cutting instrument recited in claim 15 wherein said angled region defines a bend angle in said outer tubular member and said flexible region conforms to a bend angle of up to 90°.

17. An angled rotary tissue cutting instrument for cutting anatomical tissue, comprising
an elongate angled outer tubular member having a distal end, a longitudinal internal passage, an open proximal end communicating with said passage, an angled region between said distal end and said proximal end, and an opening in said distal end communicating with said passage; and
a flexible inner member for being rotatably disposed within said outer tubular member, said inner member having a distal end, a proximal end, a tubular body between said distal end of said inner member and said proximal end of said inner member, a cutting element at said distal end of said inner member, said cutting element being exposed from said opening to cut anatomical tissue when said inner member is rotatably disposed within said outer tubular member, and a flexible region for being disposed within said angled region to transmit torque to rotate said cutting element while conforming to the configuration of said angled region when said inner member is rotated within said outer tubular member, said tubular body having a central longitudinal axis and a cylindrical wall, said flexible region comprising a plurality of intertwined helical strip members formed from portions of said cylindrical wall, said helical strip members rotating in the same direction about said axis with the same pitch and helix angle from starting ends to terminal ends of said helical strip members, said starting ends of said helical strip members being unified monolithically to a solid, rigid portion of said cylindrical wall, and said terminal ends of said helical strip members being unified monolithically to a solid, rigid portion of said cylindrical wall opposite said starting ends, said solid, rigid portions of said cylindrical wall having a wall thickness, and said portions of said cylindrical wall that form said helical strips having a wall thickness the same as said wall thickness of said solid, rigid portions.

18. The angled rotary tissue cutting instrument recited in claim 17 wherein said plurality of intertwined helical strip members comprises two intertwined helical strip members of equal width.

19. The angled rotary tissue cutting instrument recited in claim 17 wherein said plurality of intertwined helical strip members comprises three intertwined helical strip members of equal width.

20. The angled rotary tissue cutting instrument recited in claim 17 wherein said plurality of intertwined helical strip members comprises four intertwined helical strip members of equal width.
